(19) United States
(12) Patent Application Publication
(10) Pub. No.: US 2025/0121323 A1
(43) Pub. Date: Apr. 17, 2025

(54) SYSTEM AND METHOD FOR TREATING AND DISPOSING OF FLUORINATED MATERIALS

(71) Applicant: PERMA-FIX ENVIRONMENTAL SERVICES, INC., Atlanta, GA (US)

(72) Inventors: Jose Gabriel Garcia, Atlanta, GA (US); Tommy W. Yarbrough, Atlanta, GA (US); Louis F. Centofanti, Atlanta, GA (US)

(21) Appl. No.: 18/888,440

(22) Filed: Sep. 18, 2024

Related U.S. Application Data
(60) Provisional application No. 63/655,844, filed on Jun. 4, 2024.
(60) Provisional application No. 63/555,113, filed on Feb. 19, 2024.
(60) Provisional application No. 63/602,736, filed on Nov. 27, 2023.
(60) Provisional application No. 63/544,544, filed on Oct. 17, 2023.

(51) Int. Cl. B01D 53/70; B01D 53/00

(52) U.S. Cl. CPC B01D 53/70; B01D 53/005; B01D 2251/302; B01D 2251/304; B01D 2251/306; B01D 2257/2064; B01D 2257/2066

(57) ABSTRACT

A system and method for treating, degrading, and incinerating a fluorocarbon or fluorinated material, such as Freon®, with reduced emissions of gaseous perfluorinated compounds (PFCs) is provided. The method includes mixing the fluorinated material with a hydroxide base and optionally a solvent, such as polyethylene glycol ether, N-methylpyrrolidone, cyrene, and/or deionized water in a batch reactor to form a reaction mixture, also referred to as a suspension. The reaction mixture is heated to a temperature ranging from about 25° C. to about 400° C. for about 0.5 hours to about 240 hours to defluorinate the fluorinated material and produce a defluorinated waste product. More specifically, the method converts organic fluorine present in the fluorinated material, such as Freon®, to inorganic fluoride. Thus, the defluorinated waste product can be incinerated with reduced emissions of harmful gaseous PFCs.

### CROSS-REFERENCE TO RELATED APPLICATIONS

This U.S. non-provisional patent application claims the benefit of and priority to U.S. provisional patent application Ser. No. 63/544,544, filed Oct. 17, 2023; U.S. provisional patent application Ser. No. 63/602,736, filed Nov. 27, 2023; U.S. provisional patent application Ser. No. 63/555,113, filed Feb. 9, 2024; and U.S. provisional patent application Ser. No. 63/655,844, filed Jun. 4, 2024, the entire disclosures of which are hereby incorporated by reference in their entirety.

### BACKGROUND

### 1. Field of the Invention

A method for treating and disposing of fluorinated materials, such as Freon® or another fluorocarbon or halocarbon containing product, is provided. A system for treating and disposing of the fluorinated materials is also provided.

### 2. Related Art

Fluorinated materials typically include a fluorocarbon or chlorofluorocarbon. Fluorinated material can refer to a single chlorofluorinated compound (CFC), a single per- or polyfluorinated compound, or a mixture of several CFCs. These materials are stable, nonflammable, low toxicity gases or liquids, which have generally been used as refrigerants and as aerosol propellants. Chlorofluorocarbons, in particular, have been used as refrigerants, fire extinguishing agents, local anesthetics, aerosol propellants, blowing agent for foams, chemical intermediates, and heat transfer mediums. Chlorofluorocarbons have a low potential for toxicity in humans, although irritation of the eyes, skin, and respiratory tract have been observed following exposure. Direct contact with chlorofluorocarbons can result in frostbite. However, chronic toxicity has not been observed in humans or experimental animals. Some fluorinated materials, such as chlorofluorocarbons and hydrofluorocarbons, cause ozone depletion and contribute to global warming (1). Specifically, Freon®, the brand name for a variety of chemically synthetized refrigerant compounds containing chlorine and fluorine (2), is known to contribute to ozone depletion. There is no data concerning the ecotoxicology of chlorofluorocarbons. (3) (4).

It is recognized that nontoxic and nonflammable refrigerants, such as Freon® and other chlorofluorocarbons (CFCs), were commercialized in the manufacture of chlorofluoro-derivatives of methane and ethane until the 1930s. Since that time, studies on the synthesis of CFCs and their applications have progressed in many directions, such as aerosol, blowing agents for foam manufacture, fire extinguishers, cleaning solvents, and refrigerants. Due to the potential environmental and health effects, such as ozone depletion and a greenhouse effect, the use of CFCs has been reduced by international agreements since the end of the 1980s. Under a treaty known as the Montreal Protocol on Substances that Deplete the Ozone Layer, which was first established in 1987, several interim replacements for CFCs were developed in the 1990s, i.e., partially or fully fluorinated or partially chlorofluorinated alkanes and olefins, including hydrochlorofluorocarbons (HCFCs), hydrofluorocarbons (HFCs), perfluorocarbons (PFCs), hydrofluoroolefins (HFOs) and hydrochlorofluoroolefins (HCFO). (5) (6)

Some common CFCs which are known to have an atmospheric lifetime, ozone depletion potential (ODP), and global warming potential (GWP) include trichlorofluoromethane (CFC-11), dichlorodifluoromethane (CFC-12), chlorotrifluorocarbon (CFC-13), 1,1,2,2-tetrachloro-1,2-difluoroethane (CFC-112), 2,2-difluorotetrachloroethane (CFC-112a), 1,1,2-trichloro-1,2,2-trifluoroethane (CFC-113), 1,1,1-trichloro-2,2,2-trifluoroethane (CFC-113a), 1,2-dichlorotetrafluoroethane (CFC-114), 1,1-dichlorotetrafluoroethane (CFC-114a), chloropentafluoroethane (CFC-115) and 1,2-dichlorohexafluoro-cyclobutanes (R-316c). It is known that atmospheric lifetimes vary among CFCs. CFC-11 has the shortest lifetime and a lower GWP. (7) (8)

The United States Environmental Protection Agency (USEPA) CompTox database has identified over 9000 highly fluorinated substances with Chemical Abstracts Service numbers available in the global market, the majority being fluorinated polymers and fluorinated surfactants (9). These substances also contribute to the problem of ozone depletion and global warming. Methods and systems for reducing the undesirable effects of fluorinated materials are desired.

### SUMMARY

One aspect of the disclosure provides a method for treating fluorinated materials. The method comprises the steps of heating a fluorinated material and hydroxide base in a batch reactor at a temperature ranging from about 25° C. to about 400° C. for a duration of about 0.5 hours to about 240 hours to produce a defluorinated waste product.

Another aspect of the disclosure provides a method for disposing of a fluorinated material, comprising the steps of: treating the fluorinated material by heating a fluorinated material and hydroxide base in a batch reactor at a temperature ranging from about 25° C. to about 400° C. for a duration of about 0.5 hours to about 240 hours to produce a defluorinated waste product; and incinerating the defluorinated waste product.

Yet another aspect of the disclosure provides a system for treating a fluorinated material. The system comprises a batch reactor for heating a fluorinated material and a hydroxide base at a temperature ranging from about 25° C. to about 400° C. for a duration of about 0.5 hours to about 240 hours to produce a defluorinated waste product.

Another aspect of the disclosure provides a system for disposing of a fluorinated material comprising a batch reactor for heating a fluorinated material and a hydroxide base at a temperature ranging from about 25° C. to about 400° C. for a duration of about 0.5 hours to about 240 hours to produce a defluorinated waste product. The system further includes an incinerator for incinerating the defluorinated waste product.

### BRIEF DESCRIPTION OF THE DRAWINGS

Other advantages of the present invention will be readily appreciated, as the same becomes better understood by reference to the following detailed description when considered in connection with the accompanying drawing wherein:

FIG. 1 illustrates a batch system for defluorinating a fluorocarbon present in Freon® and which produces a defluorinated waste product according to an example embodiment. The following reaction occurs in the batch system: nKOH+Freon®→KF+non-fluorocarbon organic salts.

### DETAILED DESCRIPTION OF EXAMPLE EMBODIMENTS

The materials, compounds, compositions, and methods described herein may be understood more readily by reference to the following detailed description of specific aspects of the disclosed subject matter and the examples included therein.

Before the present materials, compounds, compositions, and methods are disclosed and described. It is to be understood that the aspects described below are not limited to specific methods of specific reagents, as such may, of course, vary. It is also to be understood that the terminology used herein is for the purpose of describing only and is not intended to be limiting.

Also, throughout this specification, various publications are referenced. The disclosures of these publications are hereby incorporated by reference into this application in order to more fully describe the state of the art to which the disclosed matter pertains.

In this specification and the claims that follow, reference will be made to several terms, which shall be defined to have the following meanings:

Throughout the specification and claims the word “comprise” and other forms of the word, such as “comprising” and “comprises,” means including but not limited to, and is not intended to exclude, for example, other additives, solvents, bases, components, integers, or steps. As used herein, the singular forms “a,” “an,” and “the” include plural referents unless the context clearly dictates otherwise. Thus, for example, reference to “a composition” includes mixtures of two or more such compositions. “Optional” or “optionally” means that the subsequently described event or circumstance can or cannot occur, and that the description includes instances where the event or circumstance occurs and instances where it does not. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

As used herein, the term “composition” is intended to encompass a product comprising the specified ingredients in the specified amounts, as well as any product which results, directly or indirectly, from combination of the specified ingredients in the specified amounts. Unless indicated otherwise, parts are parts by weight, temperature is in ° C. or is at ambient temperature, and pressure is at or near atmospheric. References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5 and are present in such ratio regardless of whether additional components are contained in the mixture. A weight percent (wt. %) of a component, unless specifically stated to the contrary, is based on the total weight of the formulation or composition in which the component is included. As used herein, the term “substituted” is contemplated to include all permissible substituents of inorganic base compounds.

Those people of ordinary skill in the art will appreciate that Compounds of Formula I are examples of inorganic base analogs. As used herein, “an analog of potassium hydroxide” or “analogs of potassium hydroxide” are not limited to those analog compounds represented by Formula I, and may include many additions or substitutions of elements, groups, or moieties to the chemical structure of potassium hydroxide.

M(OH)x Formula I,
wherein x is the number of hydroxy units per M valence; and
M is selected from the alkali or alkaline-earth metal groups.

The examples disclosed herein illustrate the systems, methods, and associated results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

One aspect of the disclosure relates to the composition and method for defluorination of Freon® or other fluorinated material, and thus generating inorganic fluoride in the form of a salt. Moreover, it relates to methods of reducing emissions of gaseous perfluorinated compounds (PFCs) during thermal treatment of fluorinated material. In specific aspects, the disclosed subject matter relates to the selection of materials for a greener process. The present disclosure also pertains to a method of adding a solvent to the fluorinated material and applying several heating temperatures in the degradation process. More specifically, the subject matter disclosed herein relates to a system and method that can be used for reducing emissions of gaseous perfluorinated compounds (PFCs) during thermal treatment of fluorinated material. Certain embodiments of this invention provide a composition comprising at least one solvent system and a strong base for defluorinating the fluorinated material in a batch reactor.

Example hydroxide bases include potassium hydroxide (KOH), calcium hydroxide (Ca(OH)₂), cesium hydroxide (CsOH), lithium hydroxide (LiOH), strontium hydroxide (Sr(OH)₂) and/or sodium hydroxide (NaOH). Example solvents include polyethylene glycol ethers, cyrene, and/or N-methylpyrrolidone (NMP). Water may also be present alone or optionally as a co-solvent in the batch reactor. Some of these solvents may already be present in a Freon® suspension composition.

The following reaction scheme I occurs in the batch reactor.

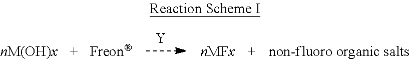

wherein;
n is the minimum amount of molar equivalents to degrade the organic fluorine in Freon®,
M is comprised of potassium, sodium, cesium, lithium, strontium or calcium;
x is the number of hydroxy (OH) of fluoride (F) units per M valence; and
Y is comprised of the list of solvents water, polyethylene glycol ethers, cyrene, and/or N-methylpyrrolidone (NMP).

In another embodiment, the compositions described above do not include addition of said solvent according to the reaction scheme II.

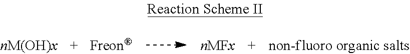

wherein;
n is the minimum amount of molar equivalents to degrade the organic fluorine in Freon®;
M is comprised of potassium, sodium, cesium, lithium, strontium or calcium; and
x is the number of hydroxy (OH) of fluoride (F) units per M valence.

The one or more Freon® is placed in the batch system containing the strong hydroxide base with or without the presence of a solvent such as water, polyethylene glycol ethers, cyrene, and/or N-methylpyrrolidone (NMP) to defluorinate the fluorocarbon(s) present in the Freon® individually or in a mixture forming a defluorinated waste product such as non-fluoro organic salts.

According to example embodiments, a mixture of two hydroxide bases is in a ratio of about 1:99 w/w % to about 99:1 w/w % is placed in the batch reactor along with the fluorinated material. According to another embodiment, a mixture of two hydroxide bases is in a ratio of about 25:75 w/w % to about 75:25 w/w % is placed in the batch reactor along with the fluorinated material. According to another embodiment, a mixture of two hydroxide bases is in a ratio of about 50:50 w/w % is placed in the batch reactor along with the fluorinated material.

According to example embodiments, the time allowed for the components to react in the batch reactor is about 0.5 hours to about 240 hours. According to another embodiment, the time allowed for the components to react in the batch reactor is about 3 hours to about 120 hours. According to another embodiment, the time allowed for the components to react in the batch reactor is about 4 hours to about 60 hours. According to another embodiment, the time allowed for the components to react in the batch reactor is about 4 hours to about 24 hours. According to another embodiment, the time allowed for the components to react in the batch is about 4 hours to about 10 hours. According to another embodiment, the time allowed for the components to react in the batch reactor is about 4 hours. According to another embodiment, the time allowed for the components to react in the batch reactor is about 8 hours. According to another embodiment, the heating temperature of the solution in the batch reactor is about 25° C. to about 300° C. According to another embodiment, the heating temperature of the solution is about 100° C. to about 300° C. According to another embodiment, the heating temperature of the solution is about 100° C. to about 200° C. According to another embodiment, the heating temperature of the solution is about 150° C. to about 200° C. According to another embodiment, the heating temperature is about 180° C.

According to an example embodiment, the hydroxide base is potassium hydroxide, the heating temperature is about 180° C. and the reaction time is of about 4 hours without the addition of a solvent. According to another embodiment, the hydroxide base is potassium hydroxide, the heating temperature is about 180° C. and the reaction time is about 8 hours without the addition of a solvent.

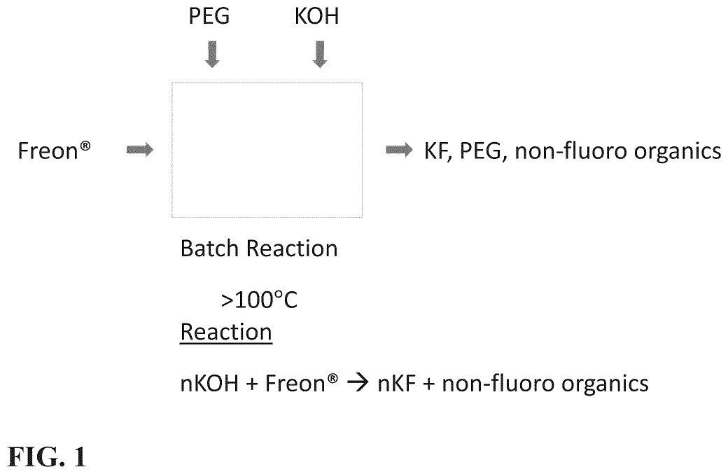

FIG. 1 illustrates a batch system for defluorinating a fluorocarbon present in Freon®, specifically, to produce a defluorinated waste product according to an example embodiment.

nM(OH)x+Freon®→nMFx+non-fluoro organic salts

As indicated above, the disclosure herein illustrates a system and method for disposing of Freon® with reduced emissions of gaseous PFC, such as CF₄ and C₂F₆. Various types of Freon® can be treated with the batch system, for example Freon-113. Although the system and method are typically applied to Freon® and discussed throughout the present disclosure, the system and method can be used to dispose of any type of fluorocarbon or fluorinated material. The system and method destroy the carbon-fluorine bonds and converts the organic fluorine present in the Freon® or other fluorinated material to inorganic fluoride.

When Freon® is the fluorinated material, the Freon® is typically maintained in the batch reactor at a temperature ranging from room temperature for several days to several weeks, or 100° C. and 200° C. for at least 2 hours, for example 3 to 5 hours, or up to 8 hours to defluorinate the Freon®s and produce a defluorinated waste product consisting of inorganic fluoride. Some types of Freon® may require higher temperatures and longer times in the reactor, for example temperatures up to but not limited to 300° C. According to other embodiments, the temperature of the batch system may be less than 100° C., for example room temperature or 50° C. up to 100° C.® When the temperature of the batch system is lower, the time required to defluorinate the Freon and produce a defluorinated waste product consisting of an inorganic fluoride is longer.

The defluorinated waste product produced may include the solvent used in the reactor, non-fluoro organic salts, and inorganic fluoride(s). The composition of the inorganic fluoride, i.e. potassium fluoride, sodium fluoride, lithium fluoride and/or calcium fluoride or combinations thereof, etc., depends on the hydroxide base or mixture of hydroxide bases used in the batch system.

After the defluorination in the batch reactor, a thermal treatment, for example incineration, may be performed on the defluorinated waste product with reduced emissions of the harmful gaseous PFCs.

According to one specific example, Freon® is placed in a batch reactor along with polyethylene glycol (PEG) and potassium hydroxide (KOH). The PEG is preferably PEG200 which has a molar mass of 190-210 g/mol and a chemical formula of H—(O—CH₂CH₂)ₙ—OH, where n=8.2 to 9.1. It is believed that the PEG200 could be replaced optionally with any known polyethylene glycol ether, or deionized water, or cyrene, or NMP or optionally no solvent added, and the KOH could be replaced with another hydroxide base comprised of but not limited to potassium, sodium, calcium, lithium, or cesium or optionally mixtures thereof, etc. According to this example, the Freon® is allowed to react in the batch system at a temperature of 180° C. to 200° C. for approximately 4 hours at ambient pressure. The resulting defluorinated waste product includes the product generated potassium fluoride (KF), PEG200, unreacted excess potassium hydroxide (KOH), and non-fluoro organic salt mixtures thereof. The chemical reaction taking place in the batch system includes:

nKOH+Freon®→nKF+non-fluoro organic salts

After the batch process, the defluorinated waste product can be thermally treated, for example by incineration, with reduced emissions of the hazardous gaseous PFCs, such as CF₄ and C₂F₆.

Before incineration, some of the components present in the defluorinated waste product can be recycled or removed and disposed of without thermal treatment. For example, according to one embodiment, the PEG200 is removed from the defluorinated waste product and recycled. The recycled PEG200 can be used in future batch systems.

### Experiment—General Procedure for the Defluorination Reaction

Freon® was treated with a hydroxide base (sodium hydroxide, potassium hydroxide or calcium hydroxide or combinations thereof in various ratios) either neat or in the presence of a solvent (a polyethylene glycol selected from ethylene glycol, diglyme, PEG200, PEG400, PEG600, PEG3350, or N-methylpyrrolidine, or cyrene or deionized water) in various ratios at 150° C. to 200° C. for 4 hours. The resulting reaction mixture is allowed to cool to room temperature then quenched by adding deionized water. The reaction material is analyzed by 19F NMR and LCMSMS.

### Representative Example 1. Solvent Assisted

In a 40 mL vial with a screwcap, PEG200 (1 equivalent w/w, 2 g) is added to crushed potassium hydroxide pellets (1 equivalent w/w, 2 g) followed by the addition of Freon-113 (1 equivalent w/w, 2 g). The vial is immersed in a pre-heated sand bath (hot plate T: 200° C.) and allowed to react for 6 hours. The resulting amber colored reaction mixture is allowed to cool to room temperature. A small blob particulate is formed which is separated from the reaction mixture by sonication for 15 minutes followed by centrifugation at 3000 rpm for 15 minutes and decanting. Both the decantate and the residue are analyzed by 19F NMR. The reaction mixture shows the presence of only inorganic potassium fluoride.

### Representative Example 2. Neat (No Solvent)

In a 40 mL vial with a screwcap, Freon-113 (1 equivalent w/w, 2 g) is added to crushed potassium hydroxide pellets (1 equivalent w/w, 2 g). The vial is immersed in a pre-heated sand bath (hot plate T: 200° C.) and allowed to react for 6 hours. The resulting reaction mixture is allowed to cool to room temperature and quenched with deionized water, followed by 19F NMR analysis.

A small blob particulate is formed which is separated from the reaction mixture by sonication for 15 minutes followed by centrifugation at 3000 rpm for 15 minutes and decanting. Both the decantate and the residue are analyzed by 19F NMR. The reaction mixture shows the presence of only inorganic potassium fluoride.

It will be appreciated by those persons skilled in the art that changes could be made to embodiments of the present invention described herein without departing from the broad inventive concept thereof. It is understood, therefore, that this invention is not limited by any particular embodiments disclosed but is intended to cover the modifications that are within the spirit and scope of the invention, as defined by the appended claims.

Obviously, many modifications and variations of the present invention are possible in light of the above teachings and may be practiced otherwise than as specifically described while within the scope of the following disclosure and claims.

### LITERATURE CITED

1. Handbook for the Montreal Protocol on Substances that Deplete the Ozone Layer—7th Edition”. United Nations Environment Programme—Ozone Secretariat. 2007. Archived from the original on 2016 May 30. Retrieved 2013 Apr. 30.).
2. Wernke, Michael J., Encyclopedia of Toxicology (Fourth Edition) Volume 4, 2024, Pages 847-852
3. Wernke, Michael J., Encyclopedia of Toxicology (Third Edition) 2014, Pages 664-666.
4. Tsai, W. T., Encyclopedia of Toxicology (Third Edition) 2014, Pages 883-884.
5. Tsai, W. T., Encyclopedia of Toxicology (Fourth Edition) 2024, Pages 917-920.
6. Sekiya et al., 2006; Rusch, 2018; Sicard and Baker, 2020)
7. Mackay et al., 2006; United Nations Environment Programme UNEP, 2018; Hodnebrog et al., 2020
8. Duenas-Laita, A., Haddad and Winchester's Clinical Management of Poisoning and Drug Overdose (Fourth Edition) 2007, Pages 1377-1384.
9. Wang, Z. et al., Environ. Int. 2013, 60, 242-8; Vierke, L. et al., Environ. Sci. Eur. 2012, 24, 16; Leung, S. C. E. et al., Sci. Total Environ. 2022, 827, 153669.

1. A method for treating a fluorinated material, comprising the steps of:
heating a fluorinated material and hydroxide base in a batch reactor at a temperature ranging from about 25° C. to about 400° C. for a duration of about 0.5 hours to about 240 hours to produce a defluorinated waste product.

2. The method of claim 1, wherein the hydroxide base includes at least one of potassium hydroxide (KOH), calcium hydroxide (Ca(OH)₂), cesium hydroxide (CsOH), lithium hydroxide (LiOH), strontium hydroxide (Sr(OH)₂) and sodium hydroxide (NaOH).

3. The method of claim 1, wherein the hydroxide base includes a mixture of at least two hydroxide bases.

4. The method of claim 1, wherein the fluorinated material includes at least one of a fluorocarbon, a chlorofluorocarbon, and a hydrofluorocarbon.

5. The method of claim 4, wherein the fluorinated material includes Freon® which contains chlorine and fluorine.

6. The method of claim 1 further including adding a solvent to the batch reactor and heating the solvent along with the fluorinated material and hydroxide base.

7. The method of claim 6, wherein the solvent includes at least one of polyethylene glycol ether, cyrene, N-methylpyrrolidone, and deionized water.

8. The method of claim 1, wherein no solvent is added to the batch reactor.

9. The method of claim 1, wherein the duration of the heating step in the batch reactor ranges from about 4 hours to about 10 hours.

10. The method of claim 1, wherein the temperature during the heating step ranges from about 150° C. to about 200° C.

11. A method for disposing of a fluorinated material, comprising the steps of:
treating the fluorinated material according to the method of claim 1; and
incinerating the defluorinated waste product.

12. A system for treating a fluorinated material, comprising:
a batch reactor for heating a fluorinated material and a hydroxide base at a temperature ranging from about 25° C. to about 400° C. for a duration of about 0.5 hours to about 240 hours to produce a defluorinated waste product.

13. The system of claim 12, wherein the hydroxide base includes at least one of potassium hydroxide (KOH), calcium hydroxide (Ca(OH)₂), cesium hydroxide (CsOH), lithium hydroxide (LiOH), strontium hydroxide (Sr(OH)₂) and sodium hydroxide (NaOH).

14. The system of claim 12, wherein the fluorinated material includes at least one of a fluorocarbon, a chlorofluorocarbon, and a hydrofluorocarbon.

15. The system of claim 14, wherein the fluorinated material includes Freon® which contains chlorine and fluorine.

16. The system of claim 12 further including a solvent in the batch reactor which is heated along with the fluorinated material and hydroxide base.

17. The system of claim 16, wherein the solvent includes at least one of polyethylene glycol ether, cyrene, N-methylpyrrolidone, and deionized water.

18. The system of claim 12, wherein the duration of the heating step in the batch reactor ranges from about 4 hours to about 10 hours.

19. The system of claim 12, wherein the temperature during the heating step ranges from about 150° C. to about 200° C.

20. A system for disposing of a fluorinated material including the batch reactor of claim 12 and further including an incinerator for incinerating the defluorinated waste product.
